(19) United States
(12) Patent Application Publication
(10) Pub. No.: US 2026/0335307 A1
(43) Pub. Date: Oct. 29, 2026

(54) ANTIMICROBIAL COMBINATIONS

(71) Applicant: Helperby Therapeutics Limited, London Greater London (GB)

(72) Inventors: Robert J H Hammond, St Andrews Fife (GB); Anthony Coates, London (GB); Yanmin Hu, London (GB)

(73) Assignee: Helperby Therapeutics Limited, London Greater London (GB)

(21) Appl. No.: 19/113,235

(22) PCT Filed: Sep. 20, 2023
(86) PCT No.: PCT/GB2023/052429

§ 371 Date: Mar. 19, 2025

(30) Foreign Application Priority Data
Sep. 20, 2022 (GB) 2213750.9

(51) Int. Cl. A61K 31/496; A61K 31/5383; A61K 31/546; A61K 31/65; A61K 38/12; A61P 31/04

(52) U.S. Cl. CPC A61K 31/496; A61K 31/5383; A61K 31/546; A61K 31/65; A61K 38/12; A61P 31/04

(57) ABSTRACT

The present invention relates to a combination comprising at least four antimicrobial agents selected from rifampicin, levofloxacin, polymyxin E, polymyxin B, doxycycline, ceftazidime, and pharmaceutically acceptable derivatives thereof. Also provided is the combination for use in the treatment of a bacterial infection.

### FIELD OF THE INVENTION

The present invention relates to a combination of at least four antimicrobial agents selected from rifampicin, levofloxacin, polymyxin E, polymyxin B, doxycycline, ceftazidime, and pharmaceutically acceptable derivatives thereof. In particular, the present invention relates to the use of such combinations to kill multiplying (i.e. log phase) microorganisms associated with bacterial infections, e.g. Gram-negative bacterial infections.

### BACKGROUND

Before the introduction of antibiotics, patients suffering from acute microbial infections (e.g. tuberculosis or pneumonia) had a low chance of survival. For example, mortality from tuberculosis was around 50%. The introduction of antimicrobial agents in the 1940s and 1950s rapidly changed this picture, and now there are approximately 100 antibiotics being used to treat different bacterial infections. This has enabled the creation of modern medicine because bacterial infections can be prevented and treated effectively in millions of patients with e.g. cancer, organ transplantation, kidney dialysis, immunosuppression and surgery.

Bacteria have, however, responded to the widespread use of antibiotics by progressively gaining resistance. Now, every country in the world has antibiotic-resistant bacteria and this resistance increases annually, thereby decreasing the effectiveness of all antibiotics. By 2040-2050, antimicrobial resistance infection deaths are predicted to exceed 10 million per year (The Review on Antimicrobial Resistance, Chaired by Jim O'Neill, May 2016).

The increase in antimicrobial resistance is expected to occur in poorer countries earlier than 2040-2050, and is already a real, practical, medical issue in Europe and the United States of America. Indeed, more than 70% of bacteria that give rise to hospital acquired infections in the USA resist at least one of the main antimicrobial agents that are typically used to fight infection (Nature Reviews, Drug Discovery, 1, 895-910 (2002)). The World Health Organization has therefore classified antimicrobial resistance as a “serious threat [that] is no longer a prediction for the future, it is happening right now in every region of the world and has the potential to affect anyone, of any age, in any country” (“Antimicrobial resistance: global report on surveillance”, The World Health Organization, April 2014). If not addressed, life expectancy could revert to pre-antibiotic levels, namely about 20 years less than todays.

A solution to the growing problem of resistant bacteria is therefore desperately needed. In essence, the medical field needs to replace the circa. 100 antibiotics with products effective against both antimicrobial resistant infections whilst avoiding the development of future antimicrobial resistance.

Current efforts to solve this problem largely concentrate on the development of new chemical entities or NCEs. Each NCE requires more than 10 years' development and costs in excess of $600 million to complete the necessary safety and clinical testing. Substantial numbers fail and hence about $3.8 billion is typically required to deliver one NCE antibiotic. Ironically, antimicrobial resistance also develops faster as the antibiotic use increases meaning that all NCEs have a limited useful life, often less than 10 years. Replacing the currently used antibiotics with NCEs would thus require approximately $3,800 billion within a 10 year period and if successful, these products would then require continued plans to replace them within their 10 year life as antimicrobial resistance develops to each one. This is clearly unsustainable, even for higher income countries.

The Applicant has identified a solution to this significant and worldwide problem. Specifically, combinations of four or more known antibiotics, surprisingly identified to have synergy against gram-negative and/or gram-positive bacteria. Such combinations have been found to kill antimicrobial-resistant bacteria, also termed drug-resistant bacteria, and avoid the development of antimicrobial resistance. The four or more known antibiotics are defined in the appended claims and described herein.

The synergy demonstrated means that the combinations have a greater biological activity than the expected additive effect of each agent at the stated dosage level. This means that lower amounts, such as 5 to 20× less antibiotic, are more effect in the combination therapy than any of the single antibiotics.

Furthermore, by combining already approved drugs (CADs), the time to approval and the cost involved is much less than NCEs. The time is, for example, about 5 years, and no more than $50M is needed to produce one new product. Since far fewer fail in development, the accumulated success/fail costs are about $130M for one CAD compared to $3.8 billion for each NCE. To replace the circa. 100 single antibiotics which are used today, the number of combinations required can be reduced by virtue of CADs having activity against a broad range of different infective species and can rejuvenate a single, unusable antibiotic due to antimicrobial resistance within a CAD with much higher activity against these antimicrobial resistant strains and avoid future antimicrobial resistance developing. As a proportion of GDP, the CAD technology moves from 4% of high income countries for NCEs to about 0.005% GDP, allowing all high, middle and even low income countries to contribute as well as benefit.

The current, global focus of exclusively repeated NCE programs with their much higher cost/time/failure potential and then short usable life, predicts effective antibiotics being exhausted within two decades, and antimicrobial resistance deaths returning us 200 years backwards. Investing even 0.1% of the NCE development costs in the CAD approach provides a practical, affordable and longer lasting solution which offers a realistic option to continue to provide useable antibiotic products—globally effective and affordable—giving hope of “Affordable Antibiotics Forever and For All”.

Over the last decade, the Applicant, a small UK company and one not-for profit organisation (GARDP) are actively developing the CAD-antibiotic combinations of existing drugs. In the present application, the Applicants have developed a new approach for replacing all antibiotics by discovering synergistic combinations of three antimicrobial agents. The combinations are active against at least Extended Spectrum Beta-Lactamase (ESBL), carbapenem-producing (CPE) and carbapenem-resistant gram-negative bacteria. Even more surprisingly, the Applicant has revealed that the concentration of each antimicrobial agent of the combination is active at a very low concentration, for example, as low as 1/16th of the agent's MICₘₒₙₒ. Thus, it may advantageously be possible to reduce any potential burden of toxicity by using lower amounts of each antimicrobial agent in the combination than e.g. for a monotherapy.

WO2015/114340 describes the use of zidovudine in combination with a polymyxin selected from colistin or polymyxin B, an anti-tuberculosis antibiotic selected from rifampicin, rifapentine or rifabutin and optionally piperine, for treating a microbial infection. WO2018/011562 describes a combination comprising zidovudine and a carbapenem, optionally with a polymyxin selected from polymyxin B and polymyxin E. The present invention does not therefore encompass these previously identified combinations of the Applicant.

Synergy is not predictable or expected when two or more actives are used in combination. Synergy in the context of antimicrobial drugs is measured in a number of ways that conform to the generally accepted opinion that “synergy is an effect greater than additive”. One of the ways to assess whether synergy has been observed is to use the “chequerboard” technique. This is a well-accepted method that leads to the generation of a value called the fractional inhibitory concentration index (FICI). Orhan et al., J. Clin. Microbiol. 2005, 43(1):140 describes the chequerboard method and analysis in the paragraph bridging pages 140-141, and explains that the FICI value is a ratio of the sum of the MIC (Minimum Inhibitory Concentration) level of each individual component alone and in the mixture.

FICI or fractional inhibitory concentration index is the sum of the FICs of each antimicrobial when used in combination. The FIC or fractional inhibitory concentration of an antimicrobial in a combination, is the MIC of the antimicrobial in the combination divided by the MIC of the same antimicrobial when used alone. Minimum inhibitory concentrations are defined in the art as the lowest concentration of an antimicrobial that will inhibit the visible growth of a microorganism after overnight incubation.

The combinations are active against resistant bacteria (see the Examples herein). The individual antimicrobial agents in these combinations are notably active in many cases at concentrations significantly below the MIC when used alone. There does not, however, appear to be a method in the art for defining synergy of a 4-mer (a combination including four antimicrobial agents) expressed as Fractional Inhibitory Concentration. There has in fact been very little previous work in this field. Hence, the present inventors devised such a method. This method is applicable for any combination with “n” antimicrobial agents.

In this method, two antibiotics were “fixed” as part of a backbone whilst the two other antibiotics were varied over a doubling concentration scale beginning at the MIC (×1) for the effective dose in monotherapy against the organism being tested. The ΣFIC was then calculated as shown below.

The ΣFIC was divided by “0.5/n” where n=number of antimicrobials in the combination to provide FIC. The inventors opted for this expression because it more closely aligns with the 2-mer synergy levels. The same FIC scale as used for 2-mers was applied: synergy is observed when FIC is <0.5. An “Additive” effect is observed when FIC is 0.5 to <1. Indifference is observed when FIC is 1 to <2. Antagonism is observed when FIC is 2 to 4.

Synergy can be expressed as “ΣFIC≤0.25×n”.

### Example of Synergy

| | MIC | MIC | | |
|---|---|---|---|---|
| | Monotherapy | combination | Ratio | |
| | (X) | therapy (Y) | Y:X | |
| Drug A | 4 | 0.75 | 0.1875 | |
| Drug B | 1 | 0.25 | 0.25 | |
| Drug C | 2 | 0.5 | 0.25 | |
| Drug D | 0.25 | 0.0625 | 0.25 | |
| | | TOTAL | 0.9375 | ΣFIC |
| | | (TOTAL/0.5)/ | 0.468 | FIC |
| | | [number | | |
| | | of drugs] | | |

The above method was used in the Examples herein.

### SUMMARY OF THE INVENTION

In one aspect the present invention provides a combination as defined in the appended claims. This combination comprises at least four antimicrobial agents selected from rifampicin, levofloxacin, polymyxin E, polymyxin B, doxycycline, ceftazidime, and pharmaceutically acceptable derivatives thereof. In various embodiments, the combination includes ceftazidime, doxycycline, or a pharmaceutically acceptable derivative thereof. This means that the remaining antimicrobial agents in the combination are selected from polymyxin E, polymyxin B, levofloxacin and rifampicin, or a pharmaceutically acceptable derivative thereof. The pharmaceutically acceptable derivative of polymyxin E may be defined as colistin sulfate, colistin methanesulfonate, or colistin methane sulfonate sodium.

In another aspect the present invention provides the combinations defined herein for use in treating a bacterial infection.

In another aspect the present invention provides a pharmaceutical composition comprising the combination defined herein and a pharmaceutically acceptable adjuvant, diluent or carrier. The pharmaceutical composition may be for use in the treatment of a bacterial infection.

In another aspect the present invention provides a product comprising at least four antimicrobial agents selected from rifampicin, levofloxacin, polymyxin E, polymyxin B, doxycycline, ceftazidime, and pharmaceutically acceptable derivatives thereof, as a combined preparation for simultaneous, separate or sequential use in treating a bacterial infection. In various embodiments, the product includes ceftazidime, doxycycline, or a pharmaceutical acceptable derivative thereof. This means that the remaining antimicrobial agents in the product are selected from polymyxin E, polymyxin B, levofloxacin and rifampicin, or a pharmaceutically acceptable derivative thereof. The pharmaceutically acceptable derivative of polymyxin E may be defined as colistin sulfate, colistin methanesulfonate, or colistin methane sulfonate sodium.

In another aspect the present invention provides the use of a first antimicrobial agent in combination with at least a second, third and fourth antimicrobial agents in the manufacture of a medicament for synergistically treating a gram-negative or gram-positive bacterial infection. The first antimicrobial agent is rifampicin or a pharmaceutically acceptable derivative thereof. The second, third and fourth antimicrobial agents are selected from levofloxacin, polymyxin E, polymyxin B, doxycycline, ceftazidime, and pharmaceutically acceptable derivatives thereof.

In another aspect the present invention provides the use of a first antimicrobial agent in combination with at least a second, third and fourth antimicrobial agents in the manufacture of a medicament for synergistically treating a gram-negative or gram-positive bacterial infection. The first antimicrobial agent is levofloxacin or a pharmaceutically acceptable derivative thereof. The second, third and fourth antimicrobial agents are selected from rifampicin, polymyxin E, polymyxin B, doxycycline, ceftazidime, and pharmaceutically acceptable derivatives thereof.

In another aspect the present invention provides the use of a first antimicrobial agent in combination with at least a second, third and fourth antimicrobial agents in the manufacture of a medicament for synergistically treating a gram-negative or gram-positive bacterial infection. The first antimicrobial agent is polymyxin E or B or a pharmaceutically acceptable derivative thereof. The second, third and fourth antimicrobial agents are selected from levofloxacin, rifampicin, doxycycline, ceftazidime, and pharmaceutically acceptable derivatives thereof.

In another aspect the present invention provides the use of a first antimicrobial agent in combination with at least a second, third and fourth antimicrobial agents in the manufacture of a medicament for synergistically treating a gram-negative or gram-positive bacterial infection. The first antimicrobial agent is doxycycline or a pharmaceutically acceptable derivative thereof. The second, third and fourth antimicrobial agents are selected from levofloxacin, polymyxin E, polymyxin B, rifampicin, ceftazidime, and pharmaceutically acceptable derivatives thereof.

In another aspect the present invention provides the use of a first antimicrobial agent in combination with at least a second, third and fourth antimicrobial agents in the manufacture of a medicament for synergistically treating a gram-negative or gram-positive bacterial infection. The first antimicrobial agent is ceftazidime or a pharmaceutically acceptable derivative thereof. The second, third and fourth antimicrobial agents are selected from levofloxacin, polymyxin E, polymyxin B, doxycycline, rifampicin, and pharmaceutically acceptable derivatives thereof.

In another aspect the present invention provides a method of treating a gram-negative or gram-positive bacterial infection, wherein the method comprises administering a pharmaceutically effective amount of a combination comprising at least four antimicrobial agents selected from rifampicin, levofloxacin, polymyxin E, polymyxin B, doxycycline, ceftazidime, and pharmaceutically acceptable derivatives thereof.

These aspects and embodiments thereof are set out in the appended independent and dependent claims. It will be appreciated that features of the dependent claims may be combined with each other and with features of the independent claims in combinations other than those explicitly set out in the claims. Furthermore, the present disclosure is not restricted to the specific embodiments set out below, but includes and contemplates any combinations of features presented herein.

The foregoing and other aspects, embodiments, features and advantages of the present disclosure will be apparent from the following detailed description. In this regard, particular sections of the description are not to be read in isolation from other sections.

### DETAILED DESCRIPTION

While various exemplary embodiments are described or suggested herein, other exemplary embodiments utilizing a variety of methods and materials similar or equivalent to those described or suggested herein are encompassed by the general inventive concepts. Those features or embodiments which are implemented conventionally may not be discussed or described in detail in the interests of brevity. It will thus be appreciated that features of apparatus, products or processes described herein which are not described in detail may be implemented in accordance with any conventional techniques for implementing such features in the respective context.

As used herein, the expressions “combination of” and “in combination with” cover separate, sequential and simultaneous administration of the agents. Unless specified to the contrary, the expressions are also intended to exclude any additional actives, e.g. “a combination comprising at least four antimicrobial agents” means that the defined agents are administered separately, sequentially or simultaneously but that no other actives are administered.

When the agents are administered sequentially, either rifampicin, levofloxacin, polymyxin E, polymyxin B, doxycycline, or ceftazidime may be administered first. When administration is simultaneous, the agents may be administered either in the same or a different pharmaceutical composition. In preferred embodiments, the agents are administered sequentially or simultaneously.

The combinations of the present invention may be used to treat gram-positive or gram-negative bacterial infections. In particular, they may be used to kill multiplying and/or clinically latent bacteria associated with such infections, preferably multiplying bacteria associated with such infections, e.g. multiplying bacteria associated with Gram-negative bacterial infections. References herein to the treatment of a bacterial infection therefore include killing multiplying and/or clinically latent microorganisms associated with such infections.

As used herein, “kill” means a loss of viability as assessed by a lack of metabolic activity.

As used herein, “clinically latent bacteria” means bacteria that is metabolically active but has a growth rate that is below the threshold of infectious disease expression. The threshold of infectious disease expression refers to the growth rate threshold below which symptoms of infectious disease in a host are absent.

The metabolic activity of clinically latent bacteria can be determined by several methods known to those skilled in the art; for example, by measuring mRNA levels in the bacteria or by determining their rate of uridine uptake. In this respect, clinically latent bacteria, when compared to bacteria under logarithmic growth conditions (in vitro or in vivo), possess reduced but still significant levels of:
(I) mRNA (e.g. from 0.0001 to 50%, such as from 1 to 30, 5 to 25 or 10 to 20%, of the level of mRNA); and/or
(II) uridine (e.g. [3H]uridine) uptake (e.g. from 0.0005 to 50%, such as from 1 to 40, 15 to 35 or 20 to 30% of the level of [3H]uridine uptake).

Clinically latent bacteria typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent bacteria are phenotypically tolerant, and as such are sensitive (in log phase) to the biostatic effects of conventional antimicrobial agents (i.e. bacteria for which the minimum inhibitory concentration (MIC) of a conventional antimicrobial is substantially unchanged); but possess drastically decreased susceptibility to drug-induced killing (e.g. bacteria for which, with any given conventional antimicrobial agent, the ratio of minimum microbiocidal concentration (e.g. minimum bactericidal concentration, MBC) to MIC is 10 or more).

In various embodiments of the invention, one or more of the aforementioned combinations is used to treat a bacterial infection, in particular the combinations may be used to kill multiplying and/or clinically latent bacteria associated with the bacterial infection. As used herein, the term “bacteria” (and derivatives thereof, such as “bacterial infection”) includes, but is not limited to, references to organisms (or infections due to organisms) of the following classes and specific types:

Gram-positive cocci, such as Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus); Streptococci (e.g. beta-haemolytic, pyogenic streptococci (such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes), microaerophilic, pyogenic streptococci (Streptococcus “milleri”, such as Strept. anginosus, Strept. constellatus constellatus, Strept. constellatus pharyngidis and Strept. intermedius), oral streptococci of the “mitis” (alpha-haemolytic-Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus); Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius); Bacillaceae, such as Bacillus anthracis, Bacillus subtilis, Bacillus thuringiensis, Bacillus stearothermophilus and Bacillus cereus;

Gram-negative cocci, such as Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri; Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvei), Erwinia (e.g. Erwinia persicinus), Morganella (e.g. Morganella morganii), Salmonella (Salmonella enterica and Salmonella typhi), Shigella (e.g. Shigella dysenteriae, Shigella flexneri, Shigella boydii and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis), Proteus (e.g. Pr. mirabilis, Pr. rettgeri and Pr. vulgaris), Providencia (e.g. Providencia alcalifaciens, Providencia rettgeri and Providencia stuartii), Serratia (e.g. Serratia marcescens and Serratia liquifaciens), and Yersinia (e.g. Yersinia enterocolitica, Yersinia pestis and Yersinia pseudotuberculosis); Helicobacter (e.g. Helicobacter pylori, Helicobacter cinaedi and Helicobacter fennelliae); Acinetobacter (e.g. A. baumanii, A. calcoaceticus, A. haemolyticus, A. johnsonii, A. junii, A. lwoffi and A. radioresistens); Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri); Bacteroides fragilis; Peptococcus (e.g. Peptococcus niger); Peptostreptococcus; Clostridium (e.g. C. perfringens, C. difficile, C. botulinum, C. tetani, C. absonum, C. argentinense, C. baratii, C. bifermentans, C. beijerinckii, C. butyricum, C. cadaveris, C. carnis, C. celatum, C. clostridioforme, C. cochlearium, C. cocleatum, C. fallax, C. ghonii, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium); Mycoplasma (e.g. M. pneumoniae, M. hominis, M. genitalium and M. urealyticum); Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilusobacterium hassicum, Mycobacterium intracellulare, Mycobacterium interjectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Haemophilus (e.g. Haemophilus influenzae, Haemophilus ducreyi, Haemophilus aegyptius, Haemophilus parainfluenzae, Haemophilus haemolyticus and Haemophilus parahaemolyticus); Actinobacillus (e.g. Actinobacillus actinomycetemcomitans, Actinobacillus equuli, Actinobacillus hominis, Actinobacillus lignieresii, Actinobacillus suis and Actinobacillus ureae); Actinomyces (e.g. Actinomyces israelii); Brucella (e.g. Brucella abortus, Brucella canis, Brucella melintensis and Brucella suis); Campylobacter (e.g. Campylobacter jejuni, Campylobacter coli, Campylobacter lari and Campylobacter fetus); Listeria monocytogenes; Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus); Erysipelothrix rhusopathiae; Corynebacteriaceae (e.g. Corynebacterium diphtheriae, Corynebacterium jeikeum and Corynebacterium urealyticum); Spirochaetaceae, such as Borrelia (e.g. Borrelia recurrentis, Borrelia burgdorferi, Borrelia afzelii, Borrelia andersonii, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum); Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis); Bordetella (e.g. Bordetella bronchiseptica, Bordetella hinzii, Bordetella holmseii, Bordetella parapertussis, Bordetella pertussis and Bordetella trematum); Nocardiaceae, such as Nocardia (e.g. Nocardia asteroides and Nocardia brasiliensis); Rickettsia (e.g. Ricksettsii or Coxiella burnetii); Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii); Moraxella catarrhalis; Cyclospora cayetanensis; Entamoeba histolytica; Giardia lamblia; Trichomonas vaginalis; Toxoplasma gondii; Stenotrophomonas maltophilia; Burkholderia stenotrophomonas; Burkholderia cepacia; Burkholderia mallei and Burkholderia pseudomallei; Francisella tularensis; Gardnerella (e.g. Gardneralla vaginalis and Gardneralla mobiluncus); Streptobacillus moniliformis; Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena); Bartonella (Bartonella bacilliformis, Bartonella clarridgeiae, Bartonella elizabethae, Bartonella henselae, Bartonella quintana and Bartonella vinsonii arupensis); Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii); Spirillium (e.g. Spirillum minus); Baceteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus); Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans); Porphyromonas (e.g. Porphyromonas asaccharolytica, Porphyromonas cangingivalis, Porphyromonas canoris, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae); Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium); Chlamydia (e.g. Chlamydia trachomatis); Cryptosporidium (e.g. C. parvum, C. hominis, C. canis, C. felis, C. meleagridis and C. muris); Chlamydophila (e.g. Chlamydophila abortus (Chlamydia psittaci), Chlamydophila pneumoniae (Chlamydia pneumoniae) and Chlamydophila psittaci (Chlamydia psittaci)); Leuconostoc (e.g. Leuconostoc citreum, Leuconostoc cremoris, Leuconostoc dextranicum, Leuconostoc lactis, Leuconostoc mesenteroides and Leuconostoc pseudomesenteroides); Gemella (e.g. Gemella bergeri, Gemella haemolysans, Gemella morbillorum and Gemella sanguinis); Aeromonas (e.g. Aeromonas hydrophila, Aeromonas caviae and Aeromonas veronii biovar sobria); and Ureaplasma (e.g. Ureaplasma parvum and Ureaplasma urealyticum).

Preferably the combinations of the invention are synergistic against gram-positive or gram-negative bacteria selected from:

Gram-negatives: Enterobacteriaceae, Enterobacter spp, Pseudomonas spp, Acinetobacter spp, Shigella spp, Salmonella spp, Burkholderia stenotrophomonas, Citrobacter spp, Serratia spp, Proteus spp, Morganella spp, Providencia spp, Haemophilus spp, Aeromonas spp, Pasteurella spp, Brucella spp, Helicobacter spp, Campylobacter spp, Franciella tularensis, Legionella spp, Vibrio spp, Neisseria spp, Mycobacterium spp, Yersinia pestis, Rickettsia spp.

Gram-positives: Staphylococcus spp, Enterococcus spp, Streptococcus spp, Bacillus anthracis.

The Gram-negatives may, for example be: Enterobacteriaceae, such as Escherichia coli; Enterobacter (e.g. Enterobacter aerogenes, Enterobacter agglomerans and Enterobacter cloacae); Citrobacter (such as Citrob. freundii and Citrob. divernis); Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri); Yersinia (e.g. Yersinia enterocolitica, Yersinia pestis and Yersinia pseudotuberculosis); Helicobacter (e.g. Helicobacter pylori, Helicobacter cinaedi and Helicobacter fennelliae); Acinetobacter (e.g. A. baumanii, A. calcoaceticus, A. haemolyticus, A. johnsonii, A. junii, A. lwoffi and A. radioresistens); Morganella (e.g. Morganella morganii); Salmonella (Salmonella enterica and Salmonella typhi); Shigella (e.g. Shigella dysenteriae, Shigella flexneri, Shigella boydii and Shigella sonnei); Klebsiella (e.g. Klebs. pneumoniae, Klebs. oxytoca, Klebs. ornitholytica, Klebs. planticola, Klebs. Klebs. ozaenae, terrigena, Klebs. granulomatis (Calymmatobacterium granulomatis) and Klebs. rhinoscleromatis); Burkholderia stenotrophomonas; Francisella tularensis; Serratia (e.g. Serratia marcescens and Serratia liquifaciens); Proteus (e.g. Pr. mirabilis, Pr. rettgeri and Pr. vulgaris); Providencia (e.g. Providencia alcalifaciens, Providencia rettgeri and Providencia stuartii); Haemophilus (e.g. Haemophilus influenzae, Haemophilus ducreyi, Haemophilus aegyptius, Haemophilus parainfluenzae, Haemophilus haemolyticus and Haemophilus parahaemolyticus); Aeromonas (e.g. Aeromonas hydrophila, Aeromonas caviae and Aeromonas veronii biovar sobria); Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis); Brucella (e.g. Brucella abortus, Brucella canis, Brucella melintensis and Brucella suis); Campylobacter (e.g. Campylobacter jejuni, Campylobacter coli, Campylobacter lari and Campylobacter fetus); Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii); Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus); Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri; Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilusobacterium hassicum, Mycobacterium intracellulare, Mycobacterium interjectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Rickettsia (e.g. Ricksettsii or Coxiella burnetii).

The Gram-positives may, for example, be Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus); Streptococci (e.g. beta-haemolytic, pyogenic streptococci (such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes), microaerophilic, pyogenic streptococci (Streptococcus “milleri”, such as Strept. anginosus, Strept. constellatus constellatus, Strept. constellatus pharyngidis and Strept. intermedius), oral streptococci of the “mitis” (alpha-haemolytic-Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus); Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius); Bacillus anthracis.

Preferably, the bacterial infections treated by the combinations described herein are Gram-negative bacterial infections. Particular Gram-negative bacteria that may be treated using a combination of the invention include:

Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris); Haemophilus influenzae; Mycobacteria, such as Mycobacterium tuberculosis; and Enterobacter (e.g. Enterobacter cloacae). Preferably, the bacteria are Enterobacteriaceae, such as Escherichia coli and Klebsiella (e.g. Klebs. pneumoniae and Klebs. oxytoca). Particularly preferred are Escherichia coli, and Klebs. pneumoniae (e.g. Klebs. pneumoniae subsp. pneumoniae).

The combination of the present invention is particularly beneficial in treating (multi)-drug-resistant ((M)DR) bacteria. With respect to Enterobacteriaceae, drug resistance most often builds up to carbapenemase i.e. carbapenemase-resistant strains and “extended spectrum β-lactamase” (ESBL) strains for example New Delhi Metallo-beta-lactamase-1 (NDM-1) resistant Klebs. pneumoniae, and NDM-1 E. coli. The combination of the present invention is also particularly effective against carbapenemase-producing Enterobacteriaceae (CPE). Other drug-resistant strains may also be used, such as colistin-resistant strains, and carbapenemase-resistant strains of bacteria other than Enterobacteriaceae including carbapenem-resistant Acinetobacter and carbapenem-resistant pseudomonas organisms carrying the blaKPC gene.

In various embodiments, the combination of the present invention is beneficial against the ESKAPE pathogens. These are six highly virulent and typically antibiotic resistant bacterial pathogens including Enterococcus faecium, Staphylococcus aureus, Klebsiella pneumoniae, Acinetobacter bacumannii, Pseudomonas aeruginosa, and Enterobacter spp. This group of Gram-positive and Gram-negative bacteria can evade or “escape” commonly used antibiotics due to their increasing multi-drug resistance. Accordingly the combination of the present invention is beneficial against (M)DR strains of the ESKAPE pathogens.

Advantageously, in various embodiments the combinations of the present invention may have a broader spectrum of activity than a monotherapy or combination of only two actives. In particular, various combinations may be efficacious against at least Acinetobacter, Pseudomonas and Enterobacteriaceae, which have been identified by the World Health Organisation as including multidrug resistant bacteria for which new antibiotics are critically needed.

It should be kept in mind that although a combination such as that claimed may initially be demonstrated to be functional in treating (M)DR strains, they can then be used in treating non-resistant strains. This is especially valuable in the context of the presently claimed combination where the primary therapy for Enterobacteriaceae, such as Escherichia coli, and Klebsiella (e.g. Klebs. pneumoniae and Klebs. oxytoca) are antimicrobial drugs that are expensive due to prevailing patent protection. The replacement of such “ethical” drugs by a combination of “generic” antibiotics is thought to be beneficial from a therapeutic perspective as well as financial/economic perspective in times where governments are seeking to reduce the cost of healthcare.

The combinations of the present invention may be used to treat infections associated with any of the above-mentioned bacterial organisms, and in particular they may be used for killing multiplying and/or clinically latent microorganisms associated with such an infection, e.g. an ESKAPE pathogen bacterial infection.

In various embodiments the combinations of the present invention are effective in treating infections caused by (1) Carbapenem-resistant E. coli, Klebsiella spp., Acinetobacter spp., Pseudomonas aeruginosa, Serratia spp, or Proteus, (2) MRSA, Vancomycin resistant Staphylococcus aureus (VRSA), Vancomycin resistant Enterococcus faecium (VRE), Clarithromycin resistant Helicobacter pylori, or Quinolone resistant Salmonella spp., or (3) penicillin resistant Streptococcus pneumoniae, Ampicillin resistant Haemophilus influenzae, or quinolone resistant Shigella spp. In various embodiments, the combinations of the present invention are effective in treating infections caused by Acinetobacter baumanii, Pseudomonas aeruginosa, or MRSA.

Particular conditions which may be treated using the combination of the present invention include tuberculosis (e.g. pulmonary tuberculosis, non-pulmonary tuberculosis (such as tuberculosis lymph glands, genito-urinary tuberculosis, tuberculosis of bone and joints, tuberculosis meningitis) and miliary tuberculosis), anthrax, abscesses, acne vulgaris, actinomycosis, asthma, bacilliary dysentery, bacterial conjunctivitis, bacterial keratitis, bacterial vaginosis, botulism, Buruli ulcer, bone and joint infections, bronchitis (acute or chronic), brucellosis, burn wounds, cat scratch fever, cellulitis, chancroid, cholangitis, cholecystitis, cutaneous diphtheria, cystic fibrosis, cystitis, diffuse panbronchiolitis, diphtheria, dental caries, diseases of the upper respiratory tract, eczema, empyema, endocarditis, endometritis, enteric fever, enteritis, epididymitis, epiglottitis, erysipelis, erysipelas, erysipeloid, erythrasma, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis (e.g. Madura foot), non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma (e.g. impetigo), Q fever, rat-bite fever, reticulosis, ricin poisoning, Ritter's disease, salmonellosis, salpingitis, septic arthritis, septic infections, septicaemia, sinusitis, skin infections (e.g. skin granulomas, impetigo, folliculitis and furunculosis), syphilis, systemic infections, tonsillitis, toxic shock syndrome, trachoma, tularaemia, typhoid, typhus (e.g. epidemic typhus, murine typhus, scrub typhus and spotted fever), urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, Pityriasis versicolor, ringworm and sporotrichosis; or infections with MSSA, MRSA, Staph. epidermidis, Strept. agalactiae, Strept. pyogenes, Escherichia coli, Klebs. pneumoniae, Klebs. oxytoca, Pr. mirabilis, Pr. rettgeri, Pr. vulgaris, Haemophilus influenzae, Enterococcus faecalis and Enterococcus faecium.

Particular conditions which may be treated using the combination of the present invention also include those which are caused by Gram-negative bacteria such as abscesses, asthma, bacilliary dysentery, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, nephritis, diffuse panbronchiolitis, dental caries, diseases of the upper respiratory tract, empyema, endocarditis, endometritis, enteric fever, enteritis, epididymitis, epiglottitis, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, listeriosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, pleural effusion, pneumonia, postoperative wound infections, postoperative gas gangrene, prostatitis, pseudo-membranous colitis, psittacosis, pyelonephritis, Q fever, Ritter's disease, salmonellosis, salpingitis, septic arthritis, septic infections, septicaemia, systemic infections, tonsillitis, trachoma, typhoid, urethritis, urinary tract infections, wound infections; or infections with, Escherichia coli, Klebs. pneumoniae, Klebs. oxytoca, Pr. mirabilis, Pr. rettgeri, Pr. vulgaris, Haemophilus influenzae, Enterococcus faecalis, Enterococcus faecium, and Enterobacter cloacae.

Preferably the combinations of the present invention are used to treat acute or complicated urinary tract infections, acute or complicated skin and soft tissue infections, intra-abdominal infections, upper respiratory tract infections, community-acquired pneumonia, hospital-acquired pneumonia, ventilator-associated pneumonia, or bloodstream infections.

It will be appreciated that references herein to “treatment” extend to prophylaxis as well as the treatment of established diseases or symptoms.

As used herein the term “pharmaceutically acceptable derivative” means: (a) pharmaceutically acceptable salts; (b) solvates (including hydrates) and/or (c) prodrugs (where appropriate).

Pharmaceutically acceptable salts of the compounds included in the combinations of the invention include suitable acid addition or base salts thereof. A review of suitable pharmaceutical salts may be found in Berge et al, J Pharm Sci, 66, 1-19 (1977).

Suitable acid addition salts include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, a-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts. Suitable base salts include metal salts, e.g. sodium, calcium, and amine salts.

For example, ceftazidime pentahydrate, colistin sulfate, polymyxin B sulfate, doxycycline hyclate (doxycycline hydrochloride hemiethanolate hemihydrate), doxycycline hydrochloride, doxycycline monohydrate, levofloxacin hemihydrate, and rifampicin N-oxide, are commercially available from Sigma Aldrich. Other suppliers are also known in the art.

As used herein the term “prodrug” means the antimicrobial compound, wherein one or more groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester formation (for example, any of those described above), wherein the reversion may be carried out be an esterase etc. Other such systems will be well known to those skilled in the art.

Polymyxin E or colistin is commercially available as a methanesulfonic acid derivative: colistimethate sodium or colistin sodium methanesulfonate (CMS). Colistimethate sodium is a prodrug. It is produced by the reaction of colistin with formaldehyde and sodium bisulfite, which leads to the addition of a sulfomethyl group to the primary amines of colistin. In aqueous solutions it undergoes hydrolysis to form a complex mixture of partially sulfomethylated derivatives and colistin.

The invention includes the use of these pharmaceutically acceptable derivatives and prodrugs. In particular, the invention includes the use of colistin and pharmaceutically acceptable derivatives thereof including colistin sulfate, colistimethate sodium and colistin sodium methanesulfonate.

The invention also includes where appropriate all enantiomers and tautomers of the compounds. The skilled person will recognise compounds that possess optical properties (one or more chiral carbon atoms) or tautomeric characteristics. The corresponding enantiomers and/or tautomers may be isolated or prepared by methods known in the art.

Some of the compounds included in the combinations of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the compounds or pharmaceutically acceptable salts thereof. An isotopic variation or a pharmaceutically acceptable salt thereof is defined as one in which at least one atom is replaced by an atom having the same atomic number but an atomic mass different from the atomic mass usually found in nature. Examples of isotopes that can be incorporated include isotopes of hydrogen, carbon, nitrogen, oxygen, phosphorus, sulphur, fluorine and chlorine such as 2H, 3H, 13C, 14C, 15N, 170, 180, 31P, 32P, 35S, 18F and 36Cl, respectively. Certain isotopic variations, for example, those in which a radioactive isotope such as 3H or 14C is incorporated, are useful in drug and/or substrate tissue distribution studies. Tritiated, i.e., 3H, and carbon-14, i.e., 14C, isotopes are particularly preferred for their ease of preparation and detectability. Further, substitution with isotopes such as deuterium, i.e., 2H, may afford certain therapeutic advantages resulting from greater metabolic stability, for example, increased in vivo half-life or reduced dosage requirements and hence may be preferred in some circumstances. Isotopic variations can generally be prepared by conventional procedures using appropriate isotopic variations of suitable reagents.

The compounds for use in the combination of the present invention, including the pharmaceutically acceptable derivatives or prodrugs thereof, are commercially available and/or can be prepared by synthesis methods known in the art. Ceftazidime, polymyxin E, polymyxin B, doxycycline, levofloxacin, doxycycline, rifampicin, ceftazidime pentahydrate, colistin sulfate, colistimethate sodium, colistin sodium methanesulfonate, polymyxin B sulfate, doxycycline hyclate (doxycycline hydrochloride hemiethanolate hemihydrate), doxycycline hydrochloride, doxycycline monohydrate, levofloxacin hemihydrate, and rifampicin N-oxide, are commercially available from Sigma Aldrich are for example available from Sigma-Aldrich®. Other commercial suppliers are known in the art.

Ceftazidime is sold under the brand name Fortaz among others, and is a third-generation cephalosporin antibiotic used in the treatment of a number of bacterial infections. It has the following chemical structure:

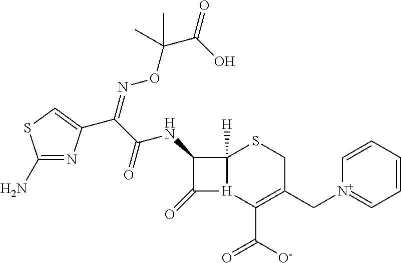

Polymyxin E is also known as colistin. It is an antibiotic medication used as a last-resort treatment for multidrug-resistant Gram-negative infections including pneumonia. These may involve bacteria such as Pseudomonas aeruginosa, Klebsiella pneumoniae, or Acinetobacter. It comes in two forms: colistimethate sodium can be injected into a vein, injected into a muscle, or inhaled, and colistin sulfate is mainly applied to the skin or taken by mouth. It has the following chemical structure:

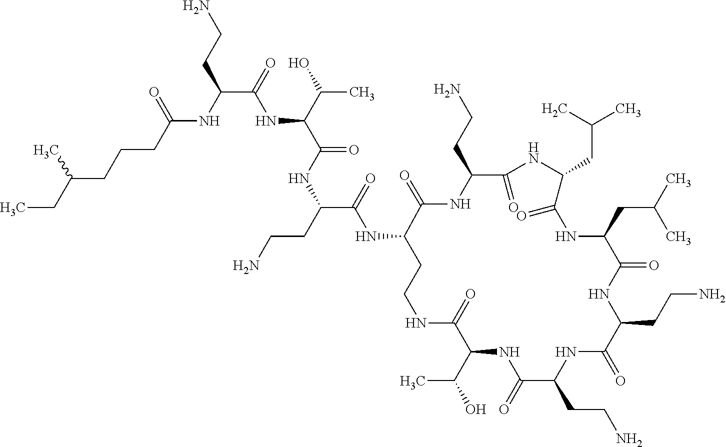

Polymyxin B, sold under the brand name Poly-Rx among others, is an antibiotic used to treat meningitis, pneumonia, sepsis, and urinary tract infections. It can be given by injection into a vein, muscle or cerebrospinal fluid, or inhaled. It has the following chemical structure:

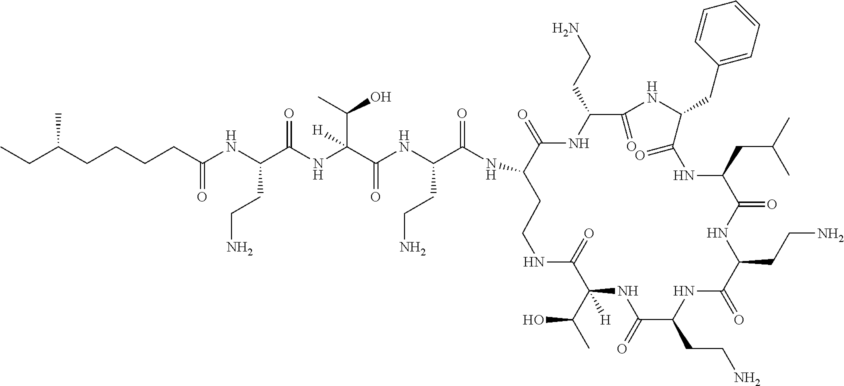 [SMILES: CC[C@H](C)CCCCC(=O)N[C@@H](CCN)C(=O)N[C@H](C(=O)N[C@@H](CCN)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@@H](CCN)NC1=O)[C@@H](C)O]

Doxycycline is a broad-spectrum tetracycline-class antibiotic used in the treatment of infections caused by bacteria and certain parasites. It is used to treat bacterial pneumonia, acne, chlamydia infections, Lyme disease, cholera, typhus, and syphilis. Doxycycline may be taken by mouth or by injection into a vein. It has the following chemical structure:

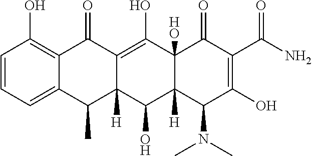 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

Levofloxacin, sold under the brand name Levaquin among others, is an antibiotic medication used to treat a number of bacterial infections including acute bacterial sinusitis, pneumonia, urinary tract infections, chronic prostatitis, and some types of gastroenteritis. It is available by mouth, intravenously, and in eye drop form. It is the(S)-isomer of ofloxacin and has the following chemical structure:

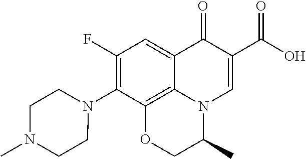 [SMILES: C[C@H]1COc2c(N3CCN(C)CC3)c(F)cc3c(=O)c(C(=O)O)cn1c23]

Rifampicin, also known as rifampin, is an ansamycin antibiotic used to treat several types of bacterial infections, including tuberculosis, Mycobacterium avium complex, leprosy, and Legionnaires' disease. Rifampicin may be given either by mouth or intravenously and has the following chemical structure:

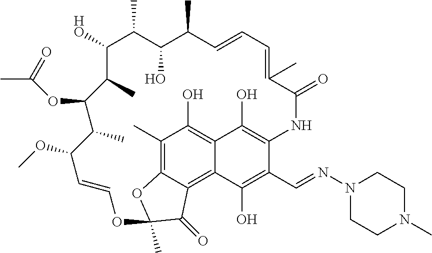 [SMILES: CO[C@H]1/C=C/O[C@@]2(C)Oc3c(C)c(O)c4c(O)c(c(/C=N/N5CCN(C)CC5)c(O)c4c3C2=O)NC(=O)/C(C)=C/C=C/[C@H](C)[C@H](O)[C@@H](C)[C@@H](O)[C@@H](C)[C@H](OC(C)=O)[C@@H]1C]

The synergistic combination of the present invention includes four antimicrobial agents. These agents are grouped in the appended claims so as to cover the exemplified combinations in the most efficient manner.

In various embodiments the at least four antimicrobial agents are selected from rifampicin, polymyxin E, polymyxin B, doxycycline, ceftazidime, and pharmaceutically acceptable derivatives thereof. Alternatively the at least four antimicrobial agents are selected from levofloxacin, polymyxin B, polymyxin E, doxycycline, ceftazidime, and pharmaceutically acceptable derivatives thereof. A further alternative is where the at least four antimicrobial agents are selected from rifampicin, levofloxacin, doxycycline, ceftazidime, and pharmaceutically acceptable derivatives thereof.

In various embodiments the at least four antimicrobial agents are rifampicin, polymyxin E, doxycycline and ceftazidime, or a pharmaceutically acceptable derivative thereof.

In various embodiments the at least four antimicrobial agents are levofloxacin, polymyxin E, doxycycline and ceftazidime, or a pharmaceutically acceptable derivative thereof.

In various embodiments the at least four antimicrobial agents are rifampicin, levofloxacin, doxycycline, ceftazidime, or a pharmaceutically acceptable derivative thereof.

Preferably the at least four antimicrobial agents include at least ceftazidime, doxycycline, or a pharmaceutically acceptable derivative thereof.

More preferably the at least four antimicrobial agents include (i) ceftazidime or a pharmaceutically acceptable derivative thereof, (ii) doxycycline or a pharmaceutically acceptable derivative thereof, (iii) polymyxin E, polymyxin B, levofloxacin, or a pharmaceutically acceptable derivative thereof, and (iv) levofloxacin, rifampicin or a pharmaceutically acceptable derivative thereof, provided that (iii) and (iv) are different.

Compounds for use according to the invention may be administered as the raw material but are preferably provided in the form of pharmaceutical compositions. The compounds may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intrathecal, intramuscular e.g. by depot and intravenous), and rectal or in a form suitable for administration by inhalation or insufflation administration. The most suitable route of administration may depend upon the condition and disorder of the patient. Preferably, the compositions of the invention are formulated for oral administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% (such as from 3 to 50%) for liquid preparations.

The concentration of each antimicrobial agent in the synergistic combination is equal to or less than the minimum inhibitory concentration in monotherapy for the bacteria against which the combination is being used (i.e. MICₘₒₙₒ). Reference herein to “MIC” should therefore be understood as MICₘₒₙₒ unless otherwise specified. Preferably the concentration of at least one of the antimicrobial agents in the synergistic combination is less than MICₘₒₙₒ, more preferably the concentration of at least two of the antimicrobial agents in the synergistic combination is less than MICₘₒₙₒ. The use of such concentrations is advantageous because it avoids toxicity issues and the reduces the possibility of antimicrobial resistance developing vs one or more of the agents in the combination.

In various embodiments, the concentration of ceftazidime is 1×MIC or less for the bacteria against which the combination is being used, the MIC being the minimum inhibitory concentration of ceftazidime when used alone against said bacteria, i.e. MICₘₒₙₒ. Preferably the concentration of ceftazidime may be 0.5×MICₘₒₙₒ or less for the bacteria against which the combination is being used. More preferably the concentration of ceftazidime is 0.25×MICₘₒₙₒ or less for the bacteria against which the combination is being used. Most preferably, the concentration of ceftazidime is 0.125×MICₘₒₙₒ or less for the bacteria against which the combination is being used. In some embodiments, the concentration of ceftazidime is as low as 0.0625×MICₘₒₙₒ-equivalent to 1/16^{th} MICₘₒₙₒ—for the bacteria against which the combination is being used. In other embodiments, the concentration of ceftazidime is as low as 0.0039×MICₘₒₙₒ-equivalent to 1/32^{nd} MICₘₒₙₒ—for the bacteria against which the combination is being used.

As an example, the ceftazidime concentration may be about 32 mg/L or less in a combination showing synergy against ESBL E. coli. In a preferred embodiment the ceftazidime concentration may be about 1 to about 32 mg/L in a combination showing synergy against ESBL E. coli.

As another example, the ceftazidime concentration may be about 32 mg/L or less in a combination showing synergy against KPC K. pneumoniae. In a preferred embodiment the ceftazidime concentration may be about 0.125 mg/L to about 32 mg/L in a combination showing synergy against KPC K. pneumoniae.

In various embodiments the concentration of doxycycline is 1×MICₘₒₙₒ or less for the bacteria against which the combination is being used. Preferably the concentration of doxycycline may be 0.5×MICₘₒₙₒ or less for the bacteria against which the combination is being used. More preferably the concentration of doxycycline may be 0.25×MICₘₒₙₒ or less for the bacteria against which the combination is being used. Most preferably the concentration of doxycycline may be 0.125×MICₘₒₙₒ or less for the bacteria against which the combination is being used.

As an example, the doxycycline concentration may be about 1 mg/L or less in a combination showing synergy against ESBL E. coli, preferably about 0.03 to about 1 mg/L.

As another example, the doxycycline concentration may be about 1 mg/L or less in a combination showing synergy against KPC K. pneumoniae, preferably about 0.03125 to about 1 mg/L.

The concentration ranges for doxycycline may be combined with those for ceftazidime. In various embodiments, ceftazidime is used at a concentration of about 1 to about 32 mg/L and doxycycline is used at a concentration of about 0.03 to about 1 mg/L in a combination showing synergy against ESBL E. coli. In various embodiments, ceftazidime is used at a concentration of about 0.125 to about 32 mg/L and doxycycline is used at a concentration of about 0.03125 to about 1 mg/L, more preferably 0.25 to about 32 mg/L in a combination showing synergy against KPC K. pneumoniae.

In various embodiments the concentration of rifampicin may be 1×MICₘₒₙₒ or less for the bacteria against which the combination is being used. Preferably the concentration of rifampicin may be 0.5×MICₘₒₙₒ or less for the bacteria against which the combination is being used. More preferably the concentration of rifampicin may be 0.25×MICₘₒₙₒ or less for the bacteria against which the combination is being used. Most preferably the concentration of rifampicin may be 0.125×MICₘₒₙₒ or less for the bacteria against which the combination is being used. For instance, in various embodiments the concentration of rifampicin can be 0.0625×MICₘₒₙₒ for the bacteria against which the combination is being used.

As an example, the rifampicin concentration may be about 4 mg/L or less in a combination showing synergy against ESBL E. coli, preferably about 0.125 to about 4 mg/L. In another example, the rifampicin concentration may be about 4 mg/L or less in a combination showing synergy against KPC K. pneumoniae, preferably about 0.125 mg/L to about 4 mg/L.

The concentration ranges for rifampicin may be combined with those for each of ceftazidime and doxycycline noted above.

In various embodiments, ceftazidime is used at a concentration of about 1 to about 32 mg/L, doxycycline is used at a concentration of about 0.03 to about 1 mg/L and rifampicin is used at a concentration of about 0.125 to about 4 mg/L in a combination showing synergy against ESBL E. coli. Preferably the ceftazidime concentration may be about 1 to about 32 mg/L, the doxycycline concentration may be about 0.06 to about 1 mg/L and the rifampicin concentration may be about 0.125 to about 4 mg/L in a combination showing synergy against ESBL E. coli.

In various embodiments, ceftazidime is used at a concentration of about 1 to about 32 mg/L, doxycycline is used at a concentration of about 0.03 to about 1 mg/L and rifampicin is used at a concentration of 1 to 4 mg/L in a combination showing synergy against ESBL E. coli.

In various embodiments, ceftazidime is used at a concentration of about 1 to about 32 mg/L, doxycycline is used at a concentration of about 0.03 to about 1 mg/L and rifampicin is used at a concentration of 0.125 to 4 mg/L in a combination showing synergy against ESBL E. coli.

In various embodiments, ceftazidime is used at a concentration of about 8 to about 32 mg/L, doxycycline is used at a concentration of about 0.03 to about 1 mg/L and rifampicin is used at a concentration of about 0.125 to about 4 mg/L in a combination showing synergy against ESBL E. coli. Preferably, ceftazidime may be used at a concentration of about 8 to about 32 mg/L, doxycycline may be used at a concentration of about 0.125 to about 1 mg/L and rifampicin is used at a concentration of about 0.125 to about 4 mg/L in a combination showing synergy against ESBL E. coli.

In various embodiments, ceftazidime is used at a concentration of about 0.125 to about 32 mg/L, doxycycline is used at a concentration of about 0.03125 to about 1 mg/L, and rifampicin is used at a concentration of about 0.125 mg/L to about 4 mg/L in a combination showing synergy against KPC K. pneumoniae. Preferably, ceftazidime is used at a concentration of about 0.125 to about 32 mg/L, doxycycline is used at a concentration of about 0.25 to about 1 mg/L, and rifampicin is used at a concentration of about 0.125 mg/L to about 4 mg/L in a combination showing synergy against KPC K. pneumoniae. More preferably, ceftazidime is used at a concentration of about 0.125 to about 32 mg/L, doxycycline is used at a concentration of about 0.25 to about 1 mg/L, and rifampicin is used at a concentration of about 0.25 mg/L to about 4 mg/L in a combination showing synergy against KPC K. pneumoniae.

The concentrations of each of ceftazidime, doxycycline and rifampicin may also be expressed as factors of MICₘₒₙₒ. For example, ceftazidime may be used at a concentration of 1×MICₘₒₙₒ or less, doxycycline may be used at a concentration of 1×MICₘₒₙₒ or less and rifampicin may be used at a concentration of 1×MICₘₒₙₒ or less. Preferably, ceftazidime may be used at a concentration of 1×MICₘₒₙₒ or less, doxycycline may be used at a concentration of 1×MICₘₒₙₒ or less and rifampicin may be used at a concentration of 0.125 to 1×MICₘₒₙₒ.

In various embodiments, ceftazidime may be used at a concentration of 0.5×MICₘₒₙₒ or less, doxycycline may be used at a concentration of 1×MICₘₒₙₒ or less, and rifampicin is used at a concentration of 1×MICₘₒₙₒ or less. Alternatively, ceftazidime may be used at a concentration of 1×MICₘₒₙₒ or less, doxycycline may be used at a concentration of 0.5x MICₘₒₙₒ or less, and rifampicin is used at a concentration of 1×MICₘₒₙₒ or less.

In various embodiments the concentration of polymyxin E or polymyxin B may be 1x MICₘₒₙₒ or less for the bacteria against which the combination is being used. Preferably the concentration of polymyxin E or polymyxin B may be 0.5×MICₘₒₙₒ or less for the bacteria against which the combination is being used. More preferably the concentration of polymyxin E or polymyxin B may be 0.25×MICₘₒₙₒ or less for the bacteria against which the combination is being used. Most preferably the concentration of polymyxin E or polymyxin B may be 0.125×MICₘₒₙₒ or less for the bacteria against which the combination is being used.

As an example, the polymyxin E or polymyxin B concentration may be about 2 mg/L or less in a combination showing synergy against ESBL E. coli, preferably about 0.06 to about 2 mg/L.

In another example, the polymyxin E or polymyxin B concentration may be about 1 mg/L or less in a combination showing synergy against KPC K. pneumoniae, preferably about 0.125 mg/L to about 1 mg/L.

The concentration ranges for polymyxin E/B may be combined with those for each of ceftazidime, doxycycline and rifampicin above.

In various embodiments, ceftazidime is used at a concentration of about 1 to about 32 mg/L, doxycycline is used at a concentration of about 0.03 to about 1 mg/L, rifampicin is used at a concentration of about 0.125 to about 4 mg/L and polymyxin E/B is used at a concentration of about 0.06 to about 2 mg/L in a combination showing synergy against ESBL E. coli. Preferably the ceftazidime concentration may be about 1 to about 32 mg/L, the doxycycline concentration may be about 0.125 to about 1 mg/L, the rifampicin concentration may be about 0.125 to about 4 mg/L and the polymyxin E/B concentration may be about 0.125 to about 2 mg/L in a combination showing synergy against ESBL E. coli.

In various embodiments ceftazidime is used at a concentration of about 8 to about 32 mg/L, doxycycline is used at a concentration of about 0.03 to about 1 mg/L, rifampicin is used at a concentration of about 0.125 to about 4 mg/L, and polymyxin E/B is used at a concentration of about 0.06 to about 2 mg/L in a combination showing synergy against ESBL E. coli. Preferably, ceftazidime may be used at a concentration of about 8 to about 32 mg/L, doxycycline may be used at a concentration of about 0.125 to about 1 mg/L, rifampicin is used at a concentration of about 0.125 to about 4 mg/L, and polymyxin E/B is used at a concentration of about 0.06 to about 2 mg/L in a combination showing synergy against ESBL E. coli.

In various embodiments ceftazidime is used at a concentration of about 1 to about 32 mg/L, doxycycline is used at a concentration of about 0.03 to about 1 mg/L, rifampicin is used at a concentration of about 1 to 4 mg/L, and polymyxin E/B is used at a concentration of about 0.06 to about 2 mg/L in a combination showing synergy against ESBL E. coli.

In various embodiments ceftazidime is used at a concentration of about 1 to about 32 mg/L, doxycycline is used at a concentration of about 0.03 to about 1 mg/L, rifampicin is used at a concentration of about 0.125 to 4 mg/L, and polymyxin E/B is used at a concentration of about 0.25 to about 2 mg/L in a combination showing synergy against ESBL E. coli. Preferably ceftazidime is used at a concentration of about 4 to about 32 mg/L, doxycycline is used at a concentration of about 0.03 to about 1 mg/L, rifampicin is used at a concentration of about 0.25 to about 4 mg/L, and polymyxin E/B is used at a concentration of about 0.25 to about 2 mg/L in a combination showing synergy against ESBL E. coli.

In various embodiments, ceftazidime is used at a concentration of about 0.125 to about 32 mg/L, doxycycline is used at a concentration of about 0.03125 to about 1 mg/L, rifampicin is used at a concentration of about 0.125 mg/L to about 4 mg/L, and polymyxin E/B is used at a concentration of about 0.125 mg/L to about 1 mg/L in a combination showing synergy against KPC K. pneumoniae. Preferably, ceftazidime is used at a concentration of about 0.125 to about 32 mg/L, doxycycline is used at a concentration of about 0.03125 to about 1 mg/L, rifampicin is used at a concentration of about 0.125 mg/L to about 4 mg/L, and polymyxin E/B is used at a concentration of about 0.125 mg/L to about 1 mg/L in a combination showing synergy against KPC K. pneumoniae. More preferably, ceftazidime is used at a concentration of about 0.125 to about 32 mg/L, doxycycline is used at a concentration of about 0.25 to about 1 mg/L, rifampicin is used at a concentration of about 0.125 mg/L to about 4 mg/L, and polymyxin E/B is used at a concentration of about 0.125 mg/L to about 1 mg/L in a combination showing synergy against KPC K. pneumoniae. Most preferably, ceftazidime is used at a concentration of about 0.125 to about 32 mg/L, doxycycline is used at a concentration of about 0.25 to about 1 mg/L, rifampicin is used at a concentration of about 0.25 mg/L to about 4 mg/L, and polymyxin E/B is used at a concentration of about 0.125 mg/L to about 1 mg/L in a combination showing synergy against KPC K. pneumoniae.

The concentrations of each of ceftazidime, doxycycline, rifampicin and polymyxin E/B may also be expressed as factors of MICₘₒₙₒ. For example, ceftazidime may be used at a concentration of 1×MICₘₒₙₒ or less, doxycycline may be used at a concentration of 1×MICₘₒₙₒ or less, rifampicin may be used at a concentration of 1×MICₘₒₙₒ or less, and polymyxin E/B may be used at a concentration of 1×MICₘₒₙₒ or less. Preferably, ceftazidime may be used at a concentration of 1×MICₘₒₙₒ or less, doxycycline may be used at a concentration of 1x MICₘₒₙₒ or less, rifampicin may be used at a concentration of 0.0625 to 1×MICₘₒₙₒ and polymyxin E/B may be used at a concentration of 0.0625 to 1×MICₘₒₙₒ. More preferably, ceftazidime may be used at a concentration of 1×MICₘₒₙₒ or less, doxycycline may be used at a concentration of 1×MICₘₒₙₒ or less, rifampicin may be used at a concentration of 0.0625 to 1×MICₘₒₙₒ and polymyxin E/B may be used at a concentration of 0.125 to 1×MICₘₒₙₒ.

In various embodiments, ceftazidime may be used at a concentration of 0.5×MICₘₒₙₒ or less, doxycycline may be used at a concentration of 1×MICₘₒₙₒ or less, and each of polymyxin E/B and rifampicin are used at a concentration of 1×MICₘₒₙₒ or less. Alternatively, ceftazidime may be used at a concentration of 1×MICₘₒₙₒ or less, doxycycline may be used at a concentration of 0.5×MICₘₒₙₒ or less, and each of polymyxin E/B and rifampicin are used at a concentration of 1×MICₘₒₙₒ or less.

In various embodiments, ceftazidime may be used at a concentration of 0.5×MICₘₒₙₒ or less, doxycycline may be used at a concentration of 1×MICₘₒₙₒ or less, rifampicin is used at a concentration of 1×MICₘₒₙₒ or less and polymyxin E/B is used at a concentration of 0.125 to 1×MICₘₒₙₒ.

In various embodiments, the concentration of levofloxacin is 1×MICₘₒₙₒ or less for the bacteria against which the combination is being used. Preferably the concentration of levofloxacin may be 0.5×MICₘₒₙₒ or less for the bacteria against which the combination is being used. More preferably the concentration of levofloxacin may be 0.25×MICₘₒₙₒ or less for the bacteria against which the combination is being used. Most preferably, the concentration of levofloxacin is 0.125×MICₘₒₙₒ or less for the bacteria against which the combination is being used.

As an example, the levofloxacin concentration may be about 8 mg/L or less in a combination showing synergy against ESBL E. coli, preferably about 0.25 to about 8 mg/L.

The concentration ranges for levofloxacin may be combined with those for each of ceftazidime and doxycycline. In various embodiments, ceftazidime is used at a concentration of about 1 to about 32 mg/L, doxycycline is used at a concentration of about 0.03 to about 1 mg/L and levofloxacin is used at a concentration of about 0.25 to about 8 mg/L in a combination showing synergy against ESBL E. coli. Preferably the ceftazidime concentration may be about 8 to about 32 mg/L, the doxycycline concentration may be about 0.03 to about 1 mg/L and the levofloxacin concentration may be about 0.25 to about 8 mg/L in a combination showing synergy against ESBL E. coli. Most preferably the ceftazidime concentration may be about 16 to about 32 mg/L, the doxycycline concentration may be about 0.03 to about 1 mg/L and the levofloxacin concentration may be about 0.25 to about 8 mg/L in a combination showing synergy against ESBL E. coli.

The concentration ranges for ceftazidime, doxycycline and levofloxacin may also be combined with those for polymyxin E/B or rifampicin as set out below.

In various embodiments, ceftazidime is used at a concentration of about 1 to about 32 mg/L, doxycycline is used at a concentration of about 0.03 to about 1 mg/L, levofloxacin is used at a concentration of about 0.25 to about 8 mg/L and polymyxin E/B is used at a concentration of about 0.06 to about 2 mg/L in a combination showing synergy against ESBL E. coli. Preferably the ceftazidime concentration may be about 4 to about 32 mg/L, the doxycycline concentration may be about 0.06 to about 1 mg/L, the levofloxacin concentration may be about 8 mg/L and the polymyxin E/B concentration may be about 0.125 to about 2 mg/L in a combination showing synergy against ESBL E. coli.

In various embodiments ceftazidime is used at a concentration of about 16 to about 32 mg/L, doxycycline is used at a concentration of about 0.03 to about 1 mg/L, levofloxacin is used at a concentration of about 0.125 to about 4 mg/L, and polymyxin E/B is used at a concentration of about 0.06 to about 2 mg/L in a combination showing synergy against ESBL E. coli.

The concentrations of each of ceftazidime, doxycycline, levofloxacin and polymyxin E/B may also be expressed as factors of MICₘₒₙₒ. For example, ceftazidime may be used at a concentration of 1×MICₘₒₙₒ or less, doxycycline may be used at a concentration of 1×MICₘₒₙₒ or less, levofloxacin may be used at a concentration of 1×MICₘₒₙₒ or less, and polymyxin E/B may be used at a concentration of 1×MICₘₒₙₒ or less. Preferably, ceftazidime may be used at a concentration of 1×MICₘₒₙₒ or less, doxycycline may be used at a concentration of 1x MICₘₒₙₒ or less, levofloxacin may be used at a concentration of 0.0625 to 1×MICₘₒₙₒ and polymyxin E/B may be used at a concentration of 0.0625 to 1×MICₘₒₙₒ.

In various embodiments, ceftazidime may be used at a concentration of 0.5×MICₘₒₙₒ to 1×MICₘₒₙₒ, doxycycline may be used at a concentration of 1×MICₘₒₙₒ or less, and each of polymyxin E/B and levofloxacin are used at a concentration of 1×MICₘₒₙₒ or less.

In various embodiments, ceftazidime is used at a concentration of about 1 to about 32 mg/L, doxycycline is used at a concentration of about 0.06 to about 1 mg/L, levofloxacin is used at a concentration of about 0.25 to about 8 mg/L and rifampicin is used at a concentration of about 0.03 to 1 mg/L in a combination showing synergy against ESBL E. coli. Preferably the ceftazidime concentration may be about 2 to about 32 mg/L, the doxycycline concentration may be about 0.06 to about 1 mg/L, the levofloxacin concentration may be about 2 to about 8 mg/L, and rifampicin is used at a concentration of about 0.03 to 1 mg/L in a combination showing synergy against ESBL E. coli. More preferably the ceftazidime concentration may be about 16 to 32 mg/L, the doxycycline concentration may be about 0.06 to about 1 mg/L, the levofloxacin concentration may be about 4 to about 8 mg/L, and rifampicin is used at a concentration of about 0.03 to 1 mg/L in a combination showing synergy against ESBL E. coli.

Alternatively the ceftazidime concentration may be about 2 to about 32 mg/L, the doxycycline concentration may be about 0.06 to about 0.5 mg/L, the levofloxacin concentration may be about 0.25 to about 8 mg/L, the doxycycline concentration may be about 0.06 to about 1 mg/L, the levofloxacin concentration may be about 2 to about 8 mg/L, and rifampicin is used at a concentration of about 0.03 to 1 mg/L in a combination showing synergy against ESBL E. coli. Preferably the ceftazidime concentration may be about 16 to about 32 mg/L, the doxycycline concentration may be about 0.06 to about 1 mg/L, the levofloxacin concentration may be about 0.25 to about 8 mg/L, and rifampicin is used at a concentration of about 0.03 to 1 mg/L in a combination showing synergy against ESBL E. coli.

The concentrations of each of ceftazidime, doxycycline, levofloxacin and rifampicin may also be expressed as factors of MICₘₒₙₒ. For example, ceftazidime may be used at a concentration of 1×MICₘₒₙₒ or less, doxycycline may be used at a concentration of 1×MICₘₒₙₒ or less, levofloxacin may be used at a concentration of 1×MICₘₒₙₒ or less, and rifampicin may be used at a concentration of 1×MICₘₒₙₒ or less. Preferably, ceftazidime may be used at a concentration of 1×MICₘₒₙₒ or less, doxycycline may be used at a concentration of 1×MICₘₒₙₒ or less, levofloxacin may be used at a concentration of 1×MICₘₒₙₒ, and rifampicin may be used at a concentration of 1×MICₘₒₙₒ or less. Alternatively, ceftazidime may be used at a concentration of 0.5 to 1×MICₘₒₙₒ, doxycycline may be used at a concentration of 1×MICₘₒₙₒ, levofloxacin is used at a concentration of 1×MICₘₒₙₒ or less, and rifampicin may be used at a concentration of 1×MICₘₒₙₒ or less.

The lower limit for the MICₘₒₙₒ ranges defined herein is not limited. Where one is not specified, it is preferably 1/512^{th} MICₘₒₙₒ, 1/256^{th} MICₘₒₙₒ, 1/128^{th} MICₘₒₙₒ, 1/64^{th} MICₘₒₙₒ, 1/32^{nd} MICₘₒₙₒ, or 0.0625 MICₘₒₙₒ. For example, “0.5×MICₘₒₙₒ or less” becomes “0.5×MICₘₒₙₒ to 0.0625 MICₘₒₙₒ.

Formulations suitable for oral administration may be presented as discrete units such as capsules, cachets or tablets (e.g. chewable tablets in particular for paediatric administration), each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

A tablet may be made by compression or molding, optionally with one or more excipients. Compressed tablets may be prepared by compressing in a suitable machine the active ingredient in a free-flowing form such as a powder or granules, optionally mixed with other conventional excipients such as binding agents (e.g. syrup, acacia, gelatin, sorbitol, tragacanth, mucilage of starch, polyvinylpyrrolidone and/or hydroxymethyl cellulose), fillers (e.g. lactose, sugar, microcrystalline cellulose, maize-starch, calcium phosphate and/or sorbitol), lubricants (e.g. magnesium stearate, stearic acid, talc, polyethylene glycol and/or silica), disintegrants (e.g. potato starch, croscarmellose sodium and/or sodium starch glycolate) and wetting agents (e.g. sodium lauryl sulphate). Molded tablets may be made by molding in a suitable machine a mixture of the powdered active ingredient with an inert liquid diluent. The tablets may be optionally coated or scored and may be formulated so as to provide controlled release (e.g. delayed, sustained, or pulsed release, or a combination of immediate release and controlled release) of the active ingredients.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use.

Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

Combinations for use according to the invention may be presented in a pack or dispenser device which may contain one or more unit dosage forms containing the active ingredients. The pack may, e.g. comprise metal or plastic foil, such as a blister pack. Where the compositions are intended for administration as three separate compositions these may be presented in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patients' supply of a pharmaceutical from a bulk supply, in that the patient always has access to the package insert contained in the patient pack, normally missing in traditional prescriptions. The inclusion of the package insert has been shown to improve patient compliance with the physician's instructions.

The administration of the combination of the invention by means of a single patient pack, or patients packs of each composition, including a package insert directing the patient to the correct use of the invention is a desirable feature of this invention.

According to a further embodiment of the present invention there is provided a patient pack comprising at least one active of the combination according to the invention and an information insert containing directions on the use of the combination of the invention. In another embodiment of the invention, there is provided a double pack comprising in association for separate administration, an antimicrobial agent, preferably having biological activity against clinically latent microorganisms, and one or more of the compounds disclosed herein preferably having biological activity against clinically latent microorganisms.

The amount of active ingredients required for use in treatment will vary with the nature of the condition being treated and the age and condition of the patient, and will ultimately be at the discretion of the attendant physician. In general however, doses employed for adult human treatment will typically be in the range of 0.02 to 5000 mg per day, preferably 1 to 1500 mg per day. The desired dose may conveniently be presented in a single dose or as divided doses administered at appropriate intervals, e.g. as two, three or more sub-doses per day.

This information would therefore be readily obtained and understood by the person skilled in the art.

### Biological Tests

Test procedures that may be employed to determine the biological (e.g. bactericidal or antimicrobial) activity of the active ingredients include those known to persons skilled in the art for determining:
(a) bactericidal activity against clinically latent bacteria; and
(b) antimicrobial activity against log phase bacteria.

In relation to (a) above, methods for determining activity against clinically latent bacteria include a determination, under conditions known to those skilled in the art (such as those described in Nature Reviews, Drug Discovery 1, 895-910 (2002), the disclosures of which are hereby incorporated by reference), of Minimum Stationary-cidal Concentration (“MSC”) or Minimum Dormicidal Concentration (“MDC”) for a test compound.

By way of example, WO2000028074 describes a suitable method of screening compounds to determine their ability to kill clinically latent microorganisms. A typical method may include the following steps:
(1) growing a bacterial culture to stationary phase;
(2) treating the stationery phase culture with one or more antimicrobial agents at a concentration and or time sufficient to kill growing bacteria, thereby selecting a phenotypically resistant sub-population;
(3) incubating a sample of the phenotypically resistant subpopulation with one or more test compounds or agents; and
(4) assessing any antimicrobial effects against the phenotypically resistant subpopulation.

According to this method, the phenotypically resistant sub-population may be seen as representative of clinically latent bacteria which remain metabolically active in vivo and which can result in relapse or onset of disease.

In relation to (b) above, methods for determining activity against log phase bacteria include a determination, under standard conditions (i.e. conditions known to those skilled in the art, such as those described in WO 2005014585, the disclosures of which document are hereby incorporated by reference), of Minimum Inhibitory Concentration (“MIC”) or Minimum Bactericidal Concentration (“MBC”) for a test compound. Specific examples of such methods are described below.

### EXAMPLES

The antimicrobial agents were sourced from commercially available sources. They were prepared by being weighed and dissolved in water, PBS, DMSO or acidified water to a final concentration from 1-10 mg/mL. Antimicrobial solutions were diluted to 10× highest concentration used in the experiment and subsequently diluted 2x over a series not exceeding 11 serial dilutions. These left the operator with a maximum of 12 different, descending concentrations of the chosen antimicrobials.

The bacteria were obtained from Ninewells Hospital and Medical School in Dundee, Scotland. They were obtained as patient strains and characterised by Vitek 2 screening. To prepare for the Examples below, the bacteria were grown overnight in Mueller Hinton Cation adjusted broth or until confluent in media with or without supplements. The bacteria were measured for OD₆₀₀ and readings of <0.25 were returned to the incubator. Bacterial cultures were diluted in media until OD₆₀₀ reading register=<0.01, this indicated that the culture was at approximately 10⁶ CFU/ml.

All data herein was produced using the same chequerboard assay.

20 uL of antibiotic A (backbone) and 20 uL of antibiotic B (backbone), at a single dilution, were pipetted into all wells of a 96-well plate to be used.

20 uL of the lowest concentration of antibiotic C (first variable) was pipetted into column 1 of the 96-well plate. The second concentration (2-fold more concentrate) was pipetted into column 2. This process was repeated until all concentrations of antibiotic B to be used were complete.

20 uL of the lowest concentration of antibiotic D (second variable) were pipetted into row A of the 96-well plate. The second concentration (2-fold more concentrate) was pipetted into row B. This process was repeated until all concentrations of antibiotic C to be used were complete.

120 uL of sterile media (BHI/MHB2) was added.

20 ul of prepared bacterial culture was added.

Plates were sealed with lids and left to incubate for 16 hours, overnight.

Plates were read at OD₆₀₀ in a 96-well plate reader. The OD values are reported for each combination below.

As a four-mer combination, two antibiotic concentrations were varied while one remained constant, the latter is the “backbone”. This method is explained above along with the calculation of the FICI and the indicative value for synergy, indifference and antagonism.

### Example 1: Synergy Between Ceftazidime, Doxycycline, Colistin and Rifampicin

The 4-mer combination of ceftazidime, doxycycline, colistin and rifampicin was tested in the assay outlined above. Colistin at 1/16^{th} MIC (0.125 mg/L) and rifampicin at 1/16^{th} MIC (0.25 mg/L) was the backbone and the combination was tested against ESBL E. coli. The highest concentration in each experiment was equal to 1×MIC for the isolate tested.

The isolate for these Examples was resistant to cephalosporins and to ceftazidime at ≥32 mg/L. The concentrations used thus fall within the dosage guidance recommended by EUCAST (the European Committee on Antimicrobial Susceptibility Testing). EUCAST prepare Breakpoint tables for interpretation of MICs and zone diameters, see e.g. version 12.

The following concentrations (mg/L) of ceftazidime and doxycycline were tested.

| | | Ceftazidime | | | | | | |
|---|---|---|---|---|---|---|---|---|
| | Mg/L | 32 | 16 | 8 | 4 | 2 | 1 | MIC |
| Doxycycline | 1 | −0.0025 | 0.001 | 0.0015 | 0 | −0.004 | −0.001 | 1 |
| | 0.5 | 0.0035 | 0.0045 | 0.0065 | 0.0025 | 0.0085 | 0.0055 | half |
| | 0.25 | 0.0045 | 0.0025 | 0.0075 | 0.0045 | 0.0035 | 0.0775 | quarter |
| | 0.125 | 0.004 | 0.0025 | 0.145 | 0.1325 | 0.18 | 0.166 | 8th |
| | 0.0625 | 0.001 | 0.0025 | 0.0005 | 0.186 | 0.142 | 0.2415 | 16th |
| | 0.03125 | 0.0015 | −0.0005 | 0.082 | 0.2215 | 0.274 | 0.2785 | 32nd |
| | MIC | 1 | half | quarter | 8th | 16th | 32nd | |

The results reported in the above table are OD600 values determined as described herein. Bold values in the above table (and similarly for the corresponding tables of the subsequent Examples) indicate bacterial growth considered to represent ineffective bacterial killing (i.e. lack of synergy).

The MIC for each drug alone and in combination was calculated according to the method outlined above.

| | MIC | MIC | | |
|---|---|---|---|---|
| | Monotherapy | combination | Ratio | |
| | (X) | therapy (Y) | Y:X | |
| Colistin | 1 | 0.125 | 0.125 | |
| Rifampicin | 2 | 0.125 | 0.0625 | |
| Doxycycline | 1 | 0.5 | 0.5 | |
| Ceftazidime | 32 | 4 | 0.125 | |
| | | TOTAL | 0.8125 | ΣFIC |
| | | (TOTAL/0.5)/ | 0.40625 | FIC |
| | | [number | | |
| | | of drugs] | | |

The ΣFIC=0.813 and FIC=0.406. This is indicative of synergy as explained hereinabove.

The 4-mer combination of ceftazidime, doxycycline, colistin and rifampicin was also tested with colistin at 1/16^{th} MIC (0.125 mg/L) and ceftazidime at ⅛^{th} MIC (4 mg/L) as the backbone against ESBL E. coli. The following concentrations (mg/L) of rifampicin (x axis) and doxycycline (y axis) were used:

| | | Rifampicin | | | | | | |
|---|---|---|---|---|---|---|---|---|
| | | 4 | 2 | 1 | 0.5 | 0.25 | 0.125 | MIC |
| Doxycycline | 1 | 0.004333 | −0.00067 | −0.00117 | −0.00067 | −0.00117 | −0.00217 | 1 |
| | 0.5 | 0.004333 | 0.000833 | −0.00067 | −0.00017 | −0.00067 | 0.002833 | half |
| | 0.25 | 0.003333 | 0.001333 | −0.00117 | 0.000333 | 0.000333 | 0.098833 | quarter |
| | 0.125 | 0.004833 | 0.000833 | −0.00067 | 0.165333 | 0.169833 | 0.168833 | 8th |
| | 0.0625 | 0.002333 | 0.000333 | −0.00067 | 0.102333 | 0.106833 | 0.094833 | 16th |
| | 0.03125 | 0.003833 | 0.003333 | 0.000333 | 0.230333 | 0.168333 | 0.173833 | 32nd |
| | MIC | 1 | half | quarter | 8th | 16th | 32nd | |

The results reported in the above table are OD600 values determined as described herein.

The MIC for each drug alone and in combination was calculated according to the method outlined above.

| | MIC | MIC | | |
|---|---|---|---|---|
| | Monotherapy | combination | Ratio | |
| | (X) | therapy (Y) | Y:X | |
| Ceftazidime | 32 | 0.125 | 0.003906 | |
| Colistin | 1 | 0.125 | 0.125 | |
| Rifampicin | 4 | 0.25 | 0.0625 | |
| Doxycycline | 1 | 0.25 | 0.25 | |
| | | TOTAL | 0.441406 | ΣFIC |
| | | (TOTAL/0.5)/ | 0.22070 | FIC |
| | | [number | | |
| | | of drugs] | | |

The ΣFIC=0.44 and FIC=0.221. This is indicative of synergy as explained hereinabove.

The 4-mer combination of ceftazidime, doxycycline, colistin and rifampicin was also tested with ceftazidime at ⅛^{th} MIC (4 mg/L) and rifampicin at 1/16^{th} MIC (0.25 mg/L) as the backbone against ESBL E. coli. The following concentrations (mg/L) of colistin and doxycycline were tested.

| | | Colistin | | | | | | |
|---|---|---|---|---|---|---|---|---|
| | | 2 | 1 | 0.5 | 0.25 | 0.125 | 0.0625 | MIC |
| DOXY | 1 | −0.02433 | −0.02383 | −0.02483 | −0.01883 | −0.02133 | −0.00833 | 1 |
| | 0.5 | −0.03233 | −0.03133 | −0.03333 | −0.03433 | −0.03333 | −0.02883 | half |
| | 0.25 | −0.03133 | −0.03083 | −0.03283 | −0.03033 | 0.023167 | 0.039167 | quarter |
| | 0.125 | −0.02783 | −0.02883 | −0.03133 | −0.02983 | 0.144667 | 0.223667 | 8th |
| | 0.0625 | −0.02983 | −0.03033 | −0.02933 | −0.03133 | 0.149167 | 0.314667 | 16th |
| | 0.03125 | −0.03083 | −0.03083 | −0.03133 | −0.02983 | 0.266667 | 0.359667 | 32nd |
| | MIC | 1 | half | quarter | 8th | 16th | 32nd | |

The results reported in the above table are OD600 values determined as described herein.

The MIC for each drug alone and in combination was calculated according to the method outlined above.

| | MIC | MIC | | |
|---|---|---|---|---|
| | Monotherapy | combination | Ratio | |
| | (X) | therapy (Y) | Y:X | |
| Ceftazidime | 32 | 4 | 0.125 | |
| Rifampicin | 4 | 0.25 | 0.0625 | |
| Colistin | 2 | 0.25 | 0.125 | |
| Doxycycline | 1 | 0.03125 | 0.03125 | |
| | | TOTAL | 0.34375 | ΣFIC |
| | | (TOTAL/0.5)/ | 0.1719 | FIC |
| | | [number | | |
| | | of drugs] | | |

The ΣFIC=0.34 and FIC=0.172. This is indicative of synergy as explained hereinabove.

Finally the 4-mer combination of ceftazidime, doxycycline, colistin and rifampicin was tested with ceftazidime at ⅛^{th} MIC (4 mg/L) and doxycycline at 1/16^{th} MIC (0.06 mg/L) as the backbone against ESBL E. coli. The following concentrations (mg/L) of colistin and rifampicin were tested:

| | | Colistin | | | | | | |
|---|---|---|---|---|---|---|---|---|
| | Mg/L | 2 | 1 | 0.5 | 0.25 | 0.125 | 0.0625 | MIC |
| RIF | 4 | −0.025 | −0.0175 | −0.021 | −0.0185 | −0.0225 | −0.027 | 1 |
| | 2 | −0.034 | −0.0325 | −0.0305 | −0.008 | −0.034 | −0.0265 | half |
| | 1 | −0.0195 | −0.0305 | −0.031 | −0.0335 | −0.0325 | 0.1395 | quarter |
| | 0.5 | −0.034 | −0.0315 | −0.029 | −0.03 | −0.033 | 0.2055 | 8th |
| | 0.25 | −0.0325 | −0.03 | −0.029 | 0.1445 | 0.2595 | 0.12 | 16th |
| | 0.125 | −0.032 | −0.0345 | −0.0315 | 0.218 | 0.1885 | 0.2905 | 32nd |
| | MIC | 1 | half | quarter | 8th | 16th | 32nd | |

The results reported in the above table are OD600 values determined as described herein.

The MIC for each drug alone and in combination was calculated according to the method outlined above.

| | MIC | MIC | | |
|---|---|---|---|---|
| | Monotherapy | combination | Ratio | |
| | (X) | therapy (Y) | Y:X | |
| Ceftazidime | 32 | 4 | 0.125 | |
| Doxycycline | 1 | 0.0625 | 0.0625 | |
| Colistin | 2 | 0.125 | 0.0625 | |
| Rifampicin | 4 | 0.5 | 0.125 | |
| | | TOTAL | 0.375 | ΣFIC |
| | | (TOTAL/0.5)/ | 0.1875 | FIC |
| | | [number | | |
| | | of drugs] | | |

The ΣFIC=0.375 and FIC=0.188. This is indicative of synergy as explained hereinabove.

It can be seen from the various tests of Example 1 how the 4-mer combination of colistin, rifampicin, doxycycline and ceftazidime is synergistic with all compounds being able to be included at concentrations below MICₘₒₙₒ.

Synergy is not a predictable result. Synergy is especially not predictable at concentrations below MIC or against MDR bacteria (ESBL E. coli); this means that the combinations are an important development in the fight against antimicrobial resistance. Surprisingly they are successful against bacteria that has an enzyme (ESBL) found in strains known not to be killed by many of the antibiotics that those skilled in the art use to treat infections.

### Example 2: Synergy Between Ceftazidime, Doxycycline, Colistin and Levofloxacin

The 4-mer combination of ceftazidime, doxycycline, colistin and levofloxacin was tested in the assay outlined above. Colistin at 1/16^{th} MIC (0.125 mg/L) and doxycyclinet 1/16^{th} MIC (0.06 mg/L) was the backbone and the combination was tested against ESBL E. coli. The highest concentration in each experiment was equal to 1×MIC for the isolate tested. The following concentrations (mg/L) of ceftazidime and levofloxacin were used.

| | | Levofloxacin | | | | | | |
|---|---|---|---|---|---|---|---|---|
| | Mg/L | 8 | 4 | 2 | 1 | 0.5 | 0.25 | MIC |
| Ceftazidime | 32 | −0.001666667 | 0.000833 | −0.00367 | −0.00267 | −0.00267 | −0.00317 | 1 |
| | 16 | −0.004666667 | −0.00317 | 0.102833 | −0.00367 | −0.00417 | −0.00217 | half |
| | 8 | 0.058333333 | 0.241833 | 0.261333 | 0.246833 | 0.265333 | 0.225833 | quarter |
| | 4 | 0.058333333 | 0.279333 | 0.207333 | 0.204833 | 0.222333 | 0.243333 | 8th |
| | 2 | 0.093833333 | 0.268333 | 0.235333 | 0.230333 | 0.228333 | 0.232333 | 16th |
| | 1 | 0.209833333 | 0.288333 | 0.241833 | 0.230833 | 0.102833 | 0.104833 | 32nd |
| | MIC | 1 | half | quarter | 8th | 16th | 32nd | |

The results reported in the above table are OD600 values determined as described herein.

The MIC for each drug alone and in combination was calculated according to the method outlined above.

| | MIC | MIC | | |
|---|---|---|---|---|
| | Monotherapy | combination | Ratio | |
| | (X) | therapy (Y) | Y:X | |
| Colistin | 1 | 0.125 | 0.125 | |
| Doxycycline | 1 | 0.0625 | 0.0625 | |
| Levofloxacin | 8 | 0.25 | 0.03125 | |
| Ceftazidime | 32 | 16 | 0.5 | |
| | | TOTAL | 0.71875 | ΣFIC |
| | | (TOTAL/0.5)/ | 0.3594 | FIC |
| | | [number | | |
| | | of drugs] | | |

The ΣFIC=0.719 and FIC=0.359. This is indicative of synergy as explained hereinabove.

### Example 3: Synergy Between Ceftazidime, Doxycycline, Rifampicin and Levofloxacin

The 4-mer combination of ceftazidime, doxycycline, rifampicin and levofloxacin was tested in the assay outlined above. Rifampicin at 1/16^{th} MIC (0.06 mg/L) and doxycycline at 1/16^{th} MIC (0.06 mg/L) was the backbone and the combination was tested against ESBL E. coli. The highest concentration in each experiment was equal to 1×MIC for the isolate tested. The following concentrations (mg/L) of ceftazidime and levofloxacin were used.

| | | levo | | | | | | |
|---|---|---|---|---|---|---|---|---|
| | | 8 | 4 | 2 | 1 | 0.5 | 0.25 | |
| ceft | 32 | −0.00917 | −0.00667 | 0.147833 | −0.00717 | −0.00717 | −0.01117 | 1 |
| | 16 | −0.01217 | −0.00867 | −0.01067 | −0.00867 | 0.075833 | −0.01317 | half |
| | 8 | −0.00767 | 0.123833 | 0.247833 | 0.211833 | 0.136833 | 0.170333 | quarter |
| | 4 | 0.019333 | 0.265333 | 0.256333 | 0.276833 | 0.290833 | 0.240833 | 8th |
| | 2 | 0.034333 | 0.251833 | 0.259833 | 0.269833 | 0.280833 | 0.236833 | 16th |
| | 1 | 0.066333 | 0.317833 | 0.266833 | 0.270333 | 0.276333 | 0.264833 | 32nd |
| | | 1 | half | quarter | 8th | 16th | 32nd | |

The results reported in the above table are OD600 values determined as described herein.

The MIC for each drug alone and in combination was calculated according to the method outlined above.

| | MIC | MIC | | |
|---|---|---|---|---|
| | Monotherapy | combination | Ratio | |
| | (X) | therapy (Y) | Y:X | |
| Rifampicin | 1 | 0.0625 | 0.0625 | |
| Doxycycline | 1 | 0.0625 | 0.0625 | |
| Levofloxacin | 8 | 0.25 | 0.03125 | |
| Ceftazidime | 32 | 16 | 0.5 | |
| | | TOTAL | 0.65625 | ΣFIC |
| | | (TOTAL/0.5)/ | 0.3281 | FIC |
| | | [number | | |
| | | of drugs] | | |

The ΣFIC=0.656 and FIC=0.328. This is indicative of synergy as explained hereinabove.

### Example 4: Synergy Between Ceftazidime (Ceft), Doxycycline (Doxy), Rifampicin (Rif) and Colistin (CSS)

The 4-mer combination of ceftazidime, doxycycline, rifampicin and colistin was tested in the assay outlined above. Ceftazidime at 1/256^{th} MIC (0.125 mg/L) and colistin at ⅛^{th} MIC (0.125 mg/L) was the backbone and the combination was tested against KPC (Klebsiella pneumoniae carbapenemase) producing K. pneumoniae. The highest concentration in each experiment was equal to 1×MIC for the isolate tested. The following concentrations (mg/L) of ceftazidime and colistin were used.

| | | Rifampicin | | | | | |
|---|---|---|---|---|---|---|---|
| | | 4 | 2 | 1 | 0.5 | 0.25 | 0.125 |
| Doxy | 1 | 0.934915 | −0.14383 | −0.25171 | −0.14383 | −0.25171 | −0.46746 |
| | 0.5 | 0.934915 | 0.179791 | −0.14383 | −0.03596 | −0.14383 | 0.611291 |
| | 0.25 | 0.719166 | 0.287666 | −0.25171 | 0.071917 | 0.071917 | 21.32327 |
| | 0.125 | 1.04279 | 0.179791 | −0.14383 | 35.67062 | 36.6415 | 36.42575 |
| | 0.0625 | 0.503416 | 0.071917 | −0.14383 | 22.07839 | 23.04926 | 20.46027 |
| | 0.03125 mg/L | 0.827041 | 0.719166 | 0.071917 | 49.69435 | 36.31787 | 37.50449 |

The results reported in the above table are reported as percentage bacterial kill values based on OD600 values relative to a positive control.

| | MIC | MIC | | |
|---|---|---|---|---|
| | Monotherapy | combination | Ratio | |
| | (X) | therapy (Y) | Y:X | |
| Ceft | 32 | 0.125 | 0.003906 | |
| CSS | 1 | 0.125 | 0.125 | |
| Rif | 4 | 0.25 | 0.0625 | |
| Doxy | 1 | 0.25 | 0.25 | |
| | | TOTAL | 0.441406 | ΣFIC |
| | | (TOTAL/0.5)/ | 0.220703 | FIC |
| | | [number | | |
| | | of drugs] | | |

The MIC for each drug alone and in combination was calculated according to the method outlined above.

The ΣFIC=0.441 and FIC=0.221. This is indicative of synergy as explained hereinabove.

The Examples support the combinations of the invention being synergistic. Synergy is not an expected result when combining antimicrobial agents and certainly not when combining four antimicrobial agents and/or against bacteria which is multi drug resistant such as ESBL strains. The Examples support the combinations of the invention being synergistic against drug-resistant bacteria and hence providing a solution to the worldwide problem of antimicrobial resistance as discussed hereinabove. This is a significant advance in the art.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A combination comprising at least four antimicrobial agents selected from rifampicin, levofloxacin, polymyxin E, polymyxin B, doxycycline, ceftazidime, and pharmaceutically acceptable derivatives thereof.

2. The combination according to claim 1, wherein the at least four antimicrobial agents are selected from rifampicin, polymyxin E, polymyxin B, doxycycline, ceftazidime, and pharmaceutically acceptable derivatives thereof.

3. The combination according to claim 1, wherein the at least four antimicrobial agents are selected from levofloxacin, polymyxin B, polymyxin E, doxycycline, ceftazidime, and pharmaceutically acceptable derivatives thereof.

4. The combination according to claim 1, wherein the at least four antimicrobial agents include ceftazidime and doxycycline, or a pharmaceutically acceptable derivative thereof, and at least two of polymyxin E, polymyxin B, rifampicin, levofloxacin, and pharmaceutically acceptable derivatives thereof.

5. The combination according to claim 4, wherein the at least four antimicrobial agents are levofloxacin, polymyxin E, doxycycline, and ceftazidime, or a pharmaceutically acceptable derivative thereof.

6. The combination according to claim 1, wherein the pharmaceutically acceptable derivative of polymyxin E is colistin sulfate, colistin methanesulfonate, or colistin methane sulfonate sodium.

7. The combination according to claim 4, wherein the at least four antimicrobial agents are rifampicin, levofloxacin, doxycycline, ceftazidime, or a pharmaceutically acceptable derivative thereof.

8. The combination according to claim 1, for use in treating a bacterial infection.

9. A pharmaceutical composition comprising the combination according to claim 1 and a pharmaceutically acceptable adjuvant, diluent or carrier.

10. The pharmaceutical composition according to claim 9, for use in the treatment of a bacterial infection.

11. The combination for use according to claim 8, wherein the use is in killing multiplying microorganisms associated with the bacterial infection.

12. The combination for use according to claim 8, wherein the infection is a gram-negative or a gram-positive bacterial infection.

13. The combination for use according to claim 8, wherein the bacterial infection is caused by Enterobacteriaceae, preferably wherein the bacterial infection is caused by E. coli or Klebsiella pneumoniae.

14. The combination for use according to claim 8, wherein the bacterial infection is caused by Acinetobacter baumanii, Pseudomonas aeruginosa, or MRSA.

15. The combination for use according to claim 8, wherein the bacterial infection is caused by a drug-resistant strain of bacteria.

16. A product comprising at least four antimicrobial agents selected from rifampicin, levofloxacin, polymyxin E, polymyxin B, doxycycline, ceftazidime, and pharmaceutically acceptable derivatives thereof, as a combined preparation for simultaneous, separate or sequential use in treating a bacterial infection.

17. The product according to claim 16, wherein the at least four antimicrobial agents are (i) rifampicin, polymyxin E, doxycycline, and ceftazidime, or a pharmaceutically acceptable derivative thereof; (ii) levofloxacin, polymyxin E, doxycycline, and ceftazidime, or a pharmaceutically acceptable derivative thereof; or (iii) rifampicin, levofloxacin, doxycycline, and ceftazidime, or a pharmaceutically acceptable derivative thereof.
